(19) United States
(12) Patent Application Publication
(10) Pub. No.: US 2023/0260663 A1
(43) Pub. Date: Aug. 17, 2023

(54) Multimodal AI-based system and device for comprehensive cardiac management

(71) Applicants: Nadia Ammirati, Del Mar, CA (US); Janko Frerk Verhey, Norten-Hardenberg (DE); Martina Verhey, Norten-Hardenberg (DE)

(72) Inventors: Nadia Ammirati, Del Mar, CA (US); Janko Frerk Verhey, Norten-Hardenberg (DE); Martina Verhey, Norten-Hardenberg (DE)

(21) Appl. No.: 18/169,388

(22) Filed: Feb. 15, 2023

Related U.S. Application Data
(60) Provisional application No. 63/310,109, filed on Feb. 15, 2022.

(51) Int. Cl. G16H 50/50; G06F 30/27

(52) U.S. Cl. CPC G16H 50/50; G06F 30/27

(57) ABSTRACT

This invention pertains to the creation of a system of a comprehensive and integrated multimodal platform utilizing artificial intelligence systems where a multitude of input data is synthesized and a personalized proposal for cardiac management is initiated. This output is mediated through a virtual platform and in addition a physical wire mesh is constructed to the specifications of the virtual mesh and that allows for a variety of applicators at different points of interest to exercise therapeutic or diagnostic interventions with feedback to the AI system. The preferred embodiment of an applicator is the Polypus device that is applied to exert an array of diagnostic and or therapeutic interventions according to different embodiments and according to the virtual and applied to varying points of interest.

### CROSS-REFERENCES TO RELATED APPLICATIONS

Continuation of application: Provisional application No 63/310,109, filed on Feb. 15, 2022.

Related applications/patents: No. 09/538,328 filed on Mar. 29, 2000, now Pat. No. 6,695,768. No. 16/968,618 filed on Feb. 5, 2019, now Pat. No. 0,046,219. No. 17/054,638 filed on Jun. 7, 2019, now Pat. No. 0,118,572. No. 17/154,119 filed on Jan. 21, 2021, now Pat. No. 0,142,695. No. 17/238,735 filed on Apr. 23, 2021, now Pat. No. 0,257,097. No. 17/072,380 filed on Oct. 16, 2019, now Pat. No. 0,125,333. No. 16/317,901 filed on Jul. 25, 2017, now Pat. No. 0,279,886.

### BACKGROUND OF THE INVENTION

Cardiovascular disease (cvd) is the leading cause of deaths worldwide. Despite significant advances in cardiac imaging and other diagnostic modalities, and the multitude of therapeutic interventions, morbidity and mortality from cardiac disease remains high.

Moreover, even after standard interventions there remains in many cases a continued decompensation of cardiac function and failure of optimization.

The current practice for diagnosis and management of cardiac disease involves decisions that are not integrated or personalized.

Many valuable information is independently processed and the value and effects of the summation interactions and interjunctions of these diagnostic and treatment modalities has not been identified.

A multiscale problem had been tackled by simple scale solutions that have surprisingly failed and been inefficient.

We are providing a complex multidimensional simulation tagged to a physical solution to comprehensively assess and manage cardiac disease.

Patients with cardiac disease have a wide range of pathologic changes.

Some of those changes include disruption of the cardiac geometry, myocardial stiffness, electrical cardiac impulse propagation changes, valvular distortion and other physical biochemical and electric variations that are complex and all combinations of the above.

Cardiac specialists have advocated a plethora of diagnostic and therapeutic modalities including angiography, imaging, electrical mapping, biochemical testing, genetic testing etc., but the combination of these therapies and the choice for a single heart had been governed by personalized patient specific heart template development of a personalized cardiac device /treatment/proposal.

A computer-aided bespoke tailoring of a therapy or combination of therapies that are specific to this heart and the potential of tailoring and applying a device that delivers the analysis of data from a multitude of modalities with various formats is the base for any cardiac management.

Human intelligence is so far needed to filter, analyze and recommend treatment from this vast array of information.

Artificial intelligence (AI) can be trained to perform various actions in devices and machines to execute decision-making processes and actions.

AI includes machine learning (ML) through which we can analyze information from data and discover novel patterns to support and enhance the treatment.

AI can be applied in single machines or modalities but overall in a set of modalities linking all physical mechanical and electric information from each modality through neural networks and synapses that modulate and control the complex decision processes.

Algorithms can enhance the role of cardiovascular data processing, mostly but not limited to imaging, by automating many tasks or calculations, finding new patterns or phenotypes in data and providing alternative diagnoses.

### Previous Work

The available art that pertains to our invention includes simple AI to address specific questions utilizing a simplified approach in vitro driven data or sampled from retrospective analysis of many patient’s information.

The modeling there exists addresses a specific procedure: cardiac pathology.

AI is available for image recognition on platforms for mapping electrical activity.

There are AI systems to address specific questions for example calcium scores or ecg performance.

The devices available are not patient specific and do not address the interdependence of the multiple variables describing all structural and functional parameters and considerations.

There has been a plethora of new cardiac devices and modeling frameworks that have been developed addressing one problem or one device utilizing a simple approach to address a complex problem.

We are recommending a comprehensive cardiac management protocol that is patient specific.

The issue we address in our invention is that the cardiac performance is a complex integration of all these variables that have been analyzed or introduced fragmented and independent of one another,

The issue we address is that small changes in one variable can induce dramatic changes in another.

For example, a mitral valve prosthesis does not address the mobility of the mitral annulus.

Another example is that management of arrythmias and that does not integrate geometric distortions integral to the global analysis and outcome of cardiac performance.

### BRIEF SUMMARY OF THE INVENTION

Our system integrates and associates all available structural and functional data through an AI processor.

It moreover addresses and probes the interdependence of one approach; one device; or one variable into the projected total performance of that individual heart.

Our system is compromised of three arms:

### First

The AI arm including the modeling that integrates all available data in one model derived from patient specific data; Also utilizing and studying available devices performances together and independently on that virtual model of the heart and the intracardiac valves and structures as well as the arterial and venous connections of the heart.

### Second

The individualized physical supporting structure created of the wire mesh model. This structure covers as a pericardial sac the heart and supports the physical functions of the heart either mechanical or electrical using additional actuators.

### Third

### The New Polypus Device Arm

We also propose a new device that acts as an agent for the AI system as well as a patient specific multifunction versatile diagnostic and therapeutic tool.

That will perform as an epicardial and Endocardial biosensor and effector and actuator of diagnosis and data harvesting from that individual heart. This could be navigated and introduced by minimally invasive access to the heart as for example implied in this application (see schematics).

That device system interaction provides another avenue for modification, testing and confirming recommended interventions also valuable in assigning and selecting the most appropriate interventions or combination of which for that specific patient

The AI system together with the testing device would recommend the specific treatment modalities and the appropriate combination of which therefore allowing a patient specific device and projecting a summation of integrated variables: electric, geometric, physical, and/or structural.

The system sequence is as follows:
A) modeling and AI processing that integrates existing AI systems together with our AI generating system
B) a virtual manipulation arm that feeds into the prediction arm of the process based on current clinical recommendations and research
C) creation of real physical representation of the wire mesh based on the individualized model
D) the Polypus that functions both as a sensor monitor and also as an effector or and affector.

That device can feed its information either virtually or in Vivo and can be used to monitor sense pace or exercise pressure deformation or correction of distortions.

For example, a patch on the surface of the aorta that angles at a certain degree and is capable of pulsating at a certain wirelessly adjustable rate.

Another example world be an epicardial balloon destined at a specific pressure to a specific targeted point pacing at a specific rate and time to correct geometric distortion at a certain specific point.

Another example would be a layered nano-membrane that allows for expansion and relaxation that could be placed outside a valve and could correct its leak. Accordingly, a specific therapeutic device could be defined personalized for that patient and with versatile adjustable pressure and size and other parameters a bespoke device could be assembled specific to that patient.

### BRIEF DESCRIPTION OF THE SEVERAL VIEWS OF THE DRAWINGS

FIG. 1: Overview: General description of the processing including data flow and it’s application for treatment/intervention. Pre-interventional data are acquired and submitted to the data-processing unit which contains both, data-processing as well as AI. Out of the data a virtual wire mesh (mesh) is generated which could be physically printed out for interventional use. The AI calculates points of interest which could be used by applicators.

FIG. 2: Input data pre-processing for AI: Description of the data processing for all modalities including an example. The process is valid for pre-, intra- and post-interventional data acquisition.

FIG. 3: The data processing consists of three possible input data sets (Pre-, Interventional (called Intra) and Post-interventional data. The input data sets are pre-processed for the use in the AI: The AI uses all data as input as well the previously stored data in the AI database to create a virtual predictive wire mesh. The virtual wire mesh is used for both, visualization and the physical representation.

FIG. 4: Finite element modelling (FEM) out of 4D volumetric input data leads to patient-specific intra- and pericardial FEM meshes. Out of these features for neuronal network in the AI are generated. The neuronal network calculates outputs for the predictive wire mesh. For optimization purposes these outputs can serve as inputs again for the neural network.

FIG. 5: Neural network calculates outputs including points of interest.

FIG. 6: Schematics of Polypus applicators

FIG. 7: A virtual heart (1) derived from patient’s 4D ultrasound data and clinical records is sourrounded by FEM modeled patient-specific virtual wire mesh (2). The virtual wire mesh covers the heart and upper ateries.

FIG. 8: Points of interest (4) are defined on the surface of the patient-specific wire mesh (3). Each point of interest (POI) has certain attributes, such as its position, an attached applicator, and function. The position and function of such applicators can be altered by an AI neural network application, as they are given to it as inputs as so called features.

FIG. 9: A physical applicator wire mesh (6) connects physical applicators (7) such as polypus devices on given POIs on the patients heart (5). The applicator wire mesh is created from a biocompatible material and might be 3D printed or manufactured by others means.

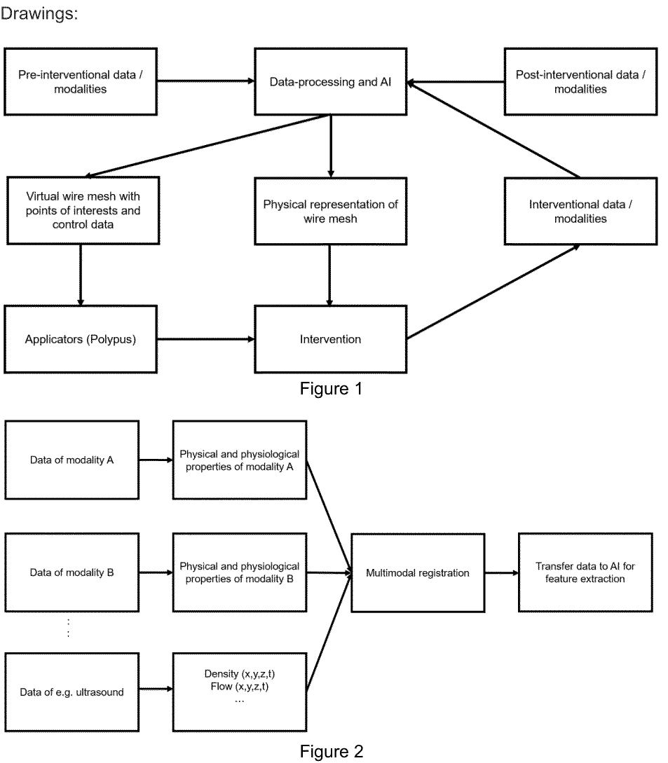

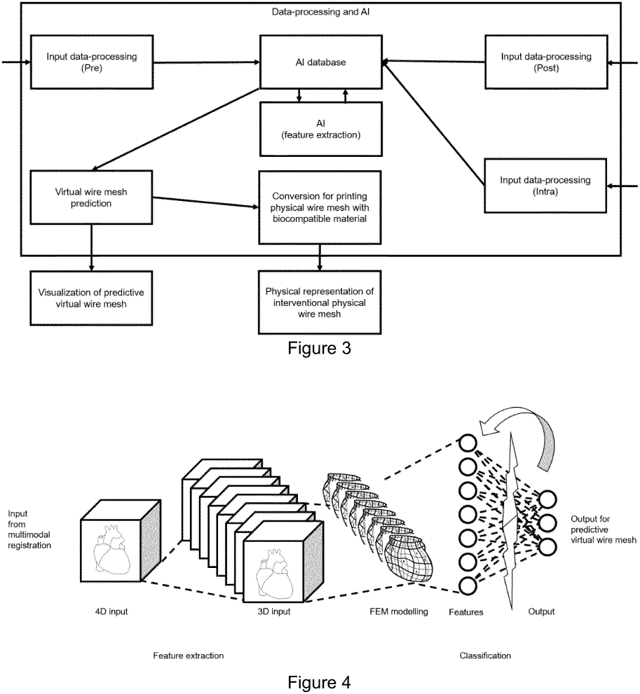

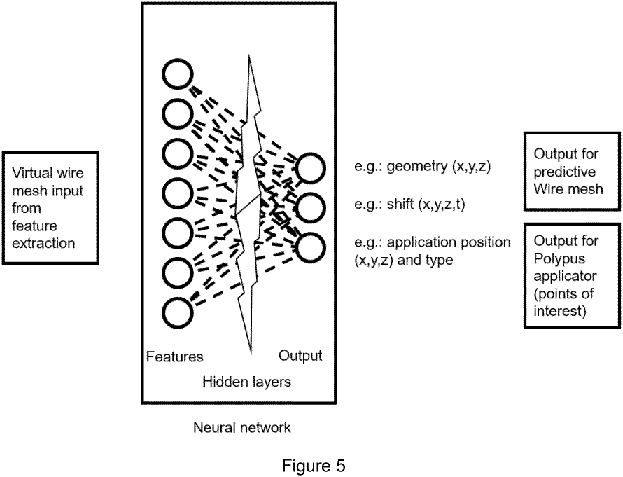

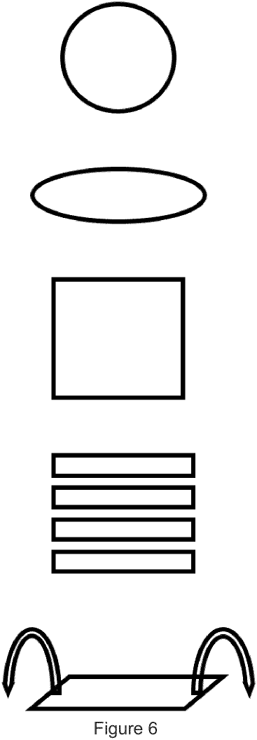

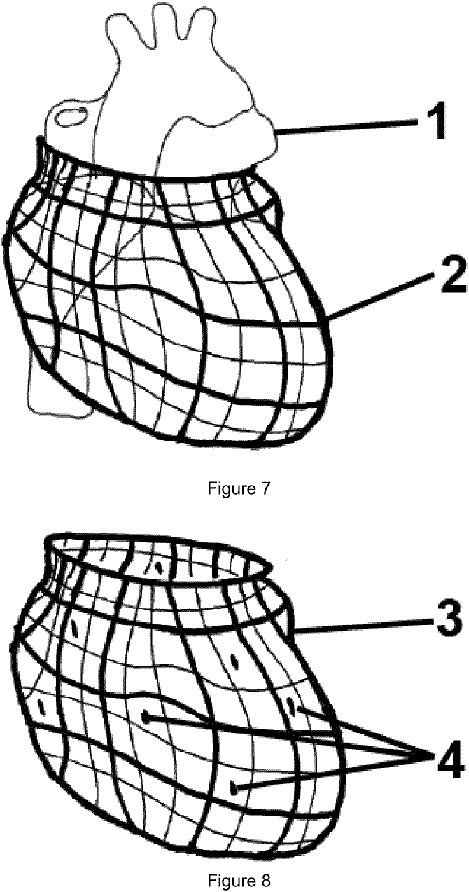

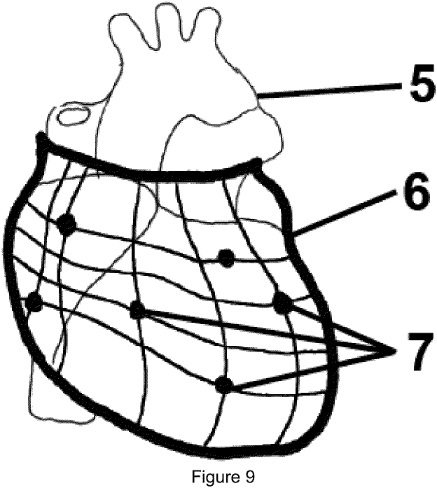

### DETAILED DESCRIPTION OF THE INVENTION

Interventions have ranged from a multitude of available valve replacement therapies, devices correcting geometric distortions, pacemakers, ablations of aberrant conduction pathways, surgical revascularization amongst other invasive and non-invasive diagnostic and therapeutic procedures.

However, the question remains for example which valve is appropriate for which patient, is there a combination of these approaches that offers the best mechanical efficiency for the function of a heart as a mechanical pump.

What is the effect of vectors and tensors inside and around the heart, how do we put all this together, how do we test it in vitro, and how do we offer the patient an integrated comprehensive answer to enhance cardiac performance?

The present invention aims to provide at a multiscale solution to comprehensively integrate and input a multitude of information through digital neural networks that relay the personalized data of all cardiac and physiologic, pathologic, biochemical, physical and spatial information from available cardiac testing and evaluation modalities as well as therapeutic interventions into a complex integrated AI system.

Embodiments of the present invention employ machine-learning algorithms to learn the complex mapping between the input parameters (e.g.: anatomical, function, and/or demographic information) and the output quantity of interest.

These methods will be used to incorporate physiological models at various scales. Although various types of physics-based models, boundary conditions, and physiological assumptions have been proposed. A common theme of mechanistic models is their use of mathematical equations to model the various physiological interactions explicitly.

The above-described methods for training a machine-learning based mapping for a patient using a trained machine-learning based mapping can be implemented on a computer using well-known computer processors, memory units, storage devices, computer software, and other components.

It also provides a physical solution to test effect the comprehensive recommendations of the system.

Embodiments of the present invention are described herein:

In preferred embodiment of that system the invention pertains to the incorporation and integration of all available information into machine-learning based assessment of effective cardiac performance given the patient personalized cardiac profile.

The present invention relates to methods and systems for machine-learning based assessment of hemodynamic indices and the development of a physical solution.

The system creates a virtual wire-mesh of the heart, the intracardiac flow patterns and the intracardiac structures and valves also the vascular connections of the heart.

A physical wire frame model of the heart is then created that can be interrogated and manipulated with available applicators. In an embodiment the Polypus device is an applicator applied to the physical wire frame created from that virtual information.

An applicator is a physical or biochemical or electrical intervention or a combination of the above. Such manipulations are virtual initially accomplished in the memory or other circuitry/ hardware of a computer system.

The computer program instructions may be stored in a storage device loaded into memory when execution of the computer program instructions is desired. Thus, the steps of the methods may be defined by the computer program instructions stored in the memory executing the computer program instructions.

An image acquisition device such as an MR scanning device, Ultrasound device, etc., can be connected wirelessly to the AI system where the image data would be channeled to the input directly as a digital neuron or through synaptic translators.

The steps of that system are as follows:
A) Incorporating information into a virtual wire mesh representation
B) Creating a virtual model of the heart
C) Creating a model of the intracardiac structures and valves
D) Inducing manipulations virtual by a set of applicators against different points on that model
E) integrates the applicator effects and summation on overall cardiac function
F) assimilates a physical wire model with characteristics derived from finite points on the virtual model
G) a Polypus device that effects and transmits those applicators to the physical wire mesh

### Phase 1

### Mathematical Modelling

Input data would include for example Echocardiography that evaluates the morphologic and flow patterns and Mechanistic models that use mathematical equations will also be used to model the physics of the blood flow in a three-dimensional frame. This system will incorporate physiological models at various scales. Although various types of physics-based models, boundary conditions, and physiological assumptions have been proposed a common theme of mechanistic models is their use of mathematical equations to model the various physiological interactions explicitly.

Embodiments of the present invention provide a data-driven and statistical methods to derive calculations from input parameters including one or more of anatomical, functional, diagnostics, molecular, and demographic information from an individual patient, or directly from medical image data.

Embodiments of the present invention employ machine-learning algorithms to learn the complex mapping between the input parameters or the input medical image data and the output hemodynamic index for example.

A set of features for the point of interest are extracted from the medical image data of the patient. of interest is determined based on the extracted set of features using a trained machine-learning based mapping.

The above-described methods for training a machine-learning based mapping for a patient using a trained machine-learning based mapping can be implemented on a computer using well-known computer processors, memory units, storage devices, computer software, and other components. A high-level block diagram of such a computer is illustrated. The present invention relates to methods and systems for machine-learning based assessment of hemodynamic indices and the development of a personalized patient specific physical solution. The embodiments of the present invention may be performed within a computer system using data stored within the computer system.

### Embodiments of the Present Invention Are Described Herein As and Are Not Limited to

Embodiments of the present invention utilize a data-driven, statistical method to calculate one or more hemodynamic indices from anatomical, functional, diagnostic, molecular, and/or demographic information from an individual patient.

Embodiments of the present invention employ machine-learning algorithms to learn the complex mapping between the input parameters (e.g., anatomical, function, and/or demographic information) and the output quantity of interest.

Unlike mechanistic model-based methods, embodiment of the present invention does not rely on an a priori assumed model describing the relationship between the inputs and the output. Instead, embodiments of the present invention determine the optimal mapping via a statistical approach using machine-learning algorithms to learn the mapping from training data. The training phase is a process, during which a database of annotated training data with is assembled.

A database from multiple patients is constructed.

In this database, is represented by several features, such as anatomical functional, diagnostic, molecular, and/or demographic measurements.

The training phase then learns or trains a mapping between the features and the values by minimizing the best fit between predictions and over the entire training database.

The prediction phase (output phase) is calculated by using the learned mapping from the training phase.

The training data can include anatomical data, functional data, and demographic data for each.

### Training Neuron

The training data is carried by a training neuron and can include anatomical data, functional data, and demographic data.

For example, the anatomical data can include: Medical imaging data obtained using one or more medical imaging modalities, such as Computed Tomography (CT), X-ray angiography, Magnetic Resonance Imaging (MRI), Ultrasound, Intra-Vascular Ultrasound (IVUS), Optical Coherence Tomography (OCT), etc.

The functional data can include functional measurements, such as blood pressure, heart rate, and ECG measurements, as well as data relating to one or more medical imaging tests for a patient, such as data from a perfusion scan (e.g., SPECT, PET, etc.) or data related to contrast agent propagation in medical images.

The demographic data can include demographic information, such as age, gender, height, and weight, etc.

The training data can also include various other types of data, such as in-vitro diagnostics data, genotype of the patient, lifestyle factors of the patient, and patient history.

Features are extracted from the training data.

The anatomical features can include anatomical measurements characterizing, as well as other anatomical measurements for associated regions such as the heart, the coronary vessel tree, the myocardium, and the aorta. And the intracardiac valves mitral tricuspid aortic and pulmonary.

Depending on the source and type of the input data, the extracted features may be binary, numerical, categorical, ordinal, binomial, interval, text-based, or combinations thereof.

According to the anatomical features extracted from medical image data can include parameters characterizing the geometry of valve attachments and or therapeutic devices proposed the shape the orientation the size and other characteristics.

It is also possible that additional parameters and characteristics can be extracted, as well, or various parameters can be combined to generate additional features.

The anatomical features extracted from the medical image data can also include parameters characterizing the morphology and other characteristics of myocardial stiffness elasticity intracardiac flow velocities vectors and intramyocardial flow velocities inclination and rotation angles in space and other spatiotemporal associations characteristics.

The anatomic features of the aorta and flow velocities and of the veins feeding the heart also the general intrathoracic cavity shape size and orientation the relative direction and position of the heart in the chest from x-ray data.

Other features extracted from the input data can also include any other parameters characterizing cardiac anatomy and function, such as end-systolic volume (ESV), end-diastolic volume (EDV), ejection fraction (EF), endocardial volume, epicardial volume, myocardial volume, trabeculae and papillary muscle volume and mass, left and right ventricular volume and mass, characteristics of contrast agent testing and attenuation amongst others.

Such features can include systolic blood pressure, diastolic blood pressure, mean arterial pressure, heart rate at rest and/or during stress, parameters derived from an ECG trace (e.g., QRS duration, R-R interval, etc.), history of heart disease, valve dysfunction, valve repair or replacement, body mass index (BMI), body surface area (BSA), weight, height, age, and sex. The features for the patient’s history may be binary, indicating that there is a history or not, or categorical, providing further indication of a category.

In addition to the anatomic and morphological features extracted from medical images, functional features may also be extracted from one or more medical imaging tests for a patient. For example, data from a perfusion scan, such as SPECT, PET, etc., may be used to extract features such as metrics characterizing relative and/or absolute tissue.

Several derived features may also be computed from the extracted features. These derived features may be represented as linear or non-linear combinations of the extracted features, which could then be used in the training database.

Furthermore, molecular information as measured by in-vitro diagnostic tests (e.g.: serum test indicating the level of myocardial damage, inflammation, etc.). The feature extraction from the medical image data for each training instance may be fully automated, semi-automated, manual, or a combination thereof.

According to our advantageous embodiment implementation, in a fully automated feature extraction approach, one or more underlying image processing algorithms to first detect the anatomical region of interest and then extract the anatomical features.

Under a semi-automated approach, some of the features may be extracted automatically, while some others may be annotated, edited, or corrected by a user. Under a manual approach, the features are annotated or measure by a user.

The feature extraction step may be performed on a medical image scanner, or on another device, such as an imaging workstation.

Once the training database is assembled with the mapping between the input features and is determined by using a machine learning algorithm.

The type of machine learning algorithm is a learned empirical model that combines the extracted features with various learned scores and weights.

non-linear relationship between image features, image context information, and anatomical object parameters such as difference in position, orientation and scale relative to current image sample will be addressed.

Boosting will operate as a feature selector, that strengthens the modeling power of weak functions and consequently accelerates the training process.

Training data may be sampled from the training neurons in order to improve computational efficiency.

The training neurons input is stored in the memory or storage of a computer system.

In a possible embodiment, a user can utilize the method to analyze the effect of different treatment scenarios, by appropriately changing the value of some features to reflect the post-treatment scenario.

As more data is collected, the training AI database containing the anatomical, physiological, and demographic measurements and/or features together with measurements may grow.

The updated AI database may then be used to re-train the machine-learning based mapping periodically.

The new instances in the training AI database may be from unseen cases (i.e., cases that have not been used for either training or prediction in the past) or from cases which were used for prediction in the past.

The training AI database may be a central database of a local database for a particular institution.

In a possible embodiment, instead of invasively hemodynamic quantities. values in the training database can be substituted by computational surrogates.

The training data may be replaced or complemented by a value numerically computed using a mechanistic modeling approach.

According to a possible embodiment, instead of using patient-specific geometries during the training phase to compute the computational surrogates for artificially generated geometries that are not based on patient-specific data - also, data from devices and other therapeutic modalities can be used.

Such geometries may be generated by varying the shape, severity, location, and number of stenoses, together with the radius and locations of main and side branches in a generic model of a device.

One advantage of using synthetically generated geometries is that it does not require the collection and processing of patient-specific data for completing the training phase, thereby saving both time and cost.

Further, there is no limit on the type of potential experimental geometries that can be generated, thereby covering a wide spectrum of vessel shapes and topology.

According to another embodiment of the present invention, instead of extracting features from the medical image data, a machine learning algorithm is applied directly on the image voxels (or sets of voxels or volume patches) to learn the association between those voxels and the hemodynamic quantity of interest.

### The Output Space

In the present application, is a value for the point of interest term uses an additive output function which aggregates a set of data.

### Phase 2 and Phase 3

The complexity of the output manifold relating the functional parameters to the input measurements can be captured and related to the physical wire frame and the ai agent in the main embodiment is the Polypus device as per the diagram.

In one embodiment the Polypus device has a center element and an effector head that has different functions and shapes according to the different embodiments and has interchangeable applicators depending on the output recommendation of the AI system.

In the present application, is a value for the point of interest term uses an additive output function which aggregates a set of data. These settings of the deep neural network can be determined experimentally.

The deep neural network layer by layer using restricted Boltzmann machines (RBM) contrastive divergences, or other auto-encoders algorithms.

Using data sets to define the weights of all layers using a gradient and back-propagation algorithms.

The foregoing detailed description is to be understood as being in every respect illustrative and exemplary, but not restrictive, and the scope of the invention disclosed herein is not to be determined from the detailed description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1.) An AI to calculate a virtual pericardiac wire mesh surrounding a virtual patient’s heart that will have nodes on biomechanical and clinically relevant points of interest (POIs) on the referenced heart. Virtual representations of Polypus devices, or any other form of measurement and or applicator devices are placed at defined POIs.

2.) A physical wire mesh that is derived from the virtual wire mesh. This is a patient-specific, biocompatible counterpart of virtual wire mesh calculated in claim 1.

3.) The devices which are biomechanical applicators, electrical applicators, or any other modifier to be attached to the physical wire mesh defined in claim 2. The apparatus for that is named Polypus. It is proposed that all POIs on the physical wire mesh have a correspondence to the virtual POIs from claim 1. This allows directed assistance.

4.) The applicators of the Polypus apparatus may vary in shape and size and functionality, e.g.:
An effector of timed pulsations
An actuator that changes resistance
An applicator as a balloon
An applicator for implantation of cells medication delivery
An applicator for hydraulic force delivery.

5.) An AI based method to alter the virtual heart’s functions by activating Polypus, applicators and measurement devices on the virtual wire mesh in order to create a predicted outcome. In addition to real-time reactions by the virtual heart collected by Polypus measurement devices, or any other form of measurement, the AI also utilizes information given by an AI database.

6.) The appliance of Polypus to the physical wire mesh to alter the functionality of the patient’s heart.
The Polypus device will be delivered by a minimally invasive approach in one embodiment the Polypus device has both sensors and pacer functions.

7.) A method to monitor the outcome of the applications on the physical wire mesh and to compare it to the predicted outcome given by the AI on the virtual wire mesh.

8.) A method to use the monitored data to improve the AI’s ability to create a set of parameters on a virtual wire mesh.
